(19) United States
(12) Patent Application Publication
(10) Pub. No.: US 2025/0339192 A1
(43) Pub. Date: Nov. 6, 2025

(54) BONE SCREW

(71) Applicant: Field Orthopaedics Pty Ltd, Teneriffe (AU)

(72) Inventors: Kau Shean Liang, Teneriffe (AU); Michael Maurer, Teneriffe (AU); Shanthan Pather, Teneriffe (AU)

(21) Appl. No.: 18/860,872

(22) PCT Filed: Apr. 27, 2023
(86) PCT No.: PCT/AU2023/050342

§ 371 Date: Oct. 28, 2024

(30) Foreign Application Priority Data
Apr. 27, 2022 (AU) 2022901105

(51) Int. Cl. A61B 17/86; A61B 17/68

(52) U.S. Cl. CPC A61B 17/863; A61B 17/8615; A61B 17/864; A61B 2017/681; A61B 2017/8655

(57) ABSTRACT

A compression screw includes an elongate shaft having a shaft length extending between a proximal and distal tip portions, the shaft having: a cannula extending coaxially through the shaft and opening through the shaft tip portion; a shaft threaded section having a length with major and minor diameters, the threaded section having screw threads positioned in a spiral pattern on an outer shaft surface and extending from a leading tip portion of the shaft to a shaft trailing portion. A buttress connects a shaft proximal portion to a threaded head with a head length with major and minor diameters with screw threads of constant pitch positioned in a spiral pattern on a head outer surface, such that M_{H}>M_{T} and m_{H}>m_{T}, the threaded head accommodates a rotational tool. A diameter of the buttress gradually increases from the proximal portion of the shaft towards the head.

### CROSS-REFERENCE TO RELATED APPLICATIONS

This application is a 35 U.S.C. § 371 National Stage patent application of PCT/AU2023/050342, filed on 27 Apr. 2023, which claims the benefit of Australian patent application no. 2022901105, filed on 27 Apr. 2022, the disclosures of which are incorporated herein by reference in their entirety.

### TECHNICAL FIELD

The present disclosure relates to bone fasteners and in particular compression screws used to bring together, and maintain compression, between two or more bones or fragments of a bone to facilitate osteosynthesis.

### BACKGROUND

Any references to methods, apparatus or documents of the prior art are not to be taken as constituting any evidence or admission that they formed, or form part of the common general knowledge.

Headless compression screws (HCSs) are commonly used to fixate small bones and articular fractures and have been used to successfully treat scaphoid, radial head, and capitellum fractures and osteotomies of the tarsal bones. With the evolution of the HCSs, several types of implants are now available. Contemporary HCSs can be divided according to shaft style into unthreaded, fully threaded, or partially threaded shaft designs. The first HCS, the Herbert compression screw (Zimmer, Warsaw, IN. USA) was introduced in the early 1980s and consists of a threadless central shaft with threads of different pitch at either end (unthreaded shaft; FIG. 1(a)). The Mini-Acutrak 2 screw (Acumed, Beaverton, OR, USA), a second-generation HCS, has a fully threaded variable-pitch design (fully threaded shaft; FIG. 1(b)). The headless reduction (HLR) screw (A Plus Biotechnology Co., Ltd., New Taipei City, Taiwan) has a fully threaded design, but two thread runouts are included in the middle shaft (partially threaded shaft; FIG. 1(c)).

By definition, the most commonly used compression screw is the Herbert Screw which comprises two sets of threads distally and proximally, separated by a smooth shaft. The leading threads have a greater pitch than the trailing threads; which allows fragments to be drawn together as trailing threads enter bone. Maximum interfragmentary compression is only achieved when the trailing threads are advanced completely within bone.

While headless screws have been well known for a few years now, there are a few disadvantages associated with headless screws. One design flaw is that many of the presently known headless screws do not initiate across the fracture site until the head of the screw is buried into the bone. This limits compressive forces to the length of the threads of the head of the screw. The small size of bones in the foot and ankle makes this issue particularly evident. It would therefore be desirable to provide an improved compression screw that can provide some of the advantages of a headless compression screw whilst still addressing at least some of the shortcomings of currently known compression screws.

### SUMMARY

In an aspect, the disclosure provides a compression or non-compression screw comprising:
an elongate shaft having a shaft length (L1) extending between a proximal portion and a distal tip portion, the shaft comprising:
   a threaded section of the shaft having a length (L2) with a major diameter (M_{T}) and a minor diameter (mₜ), the threaded section having one or more screw threads positioned in a spiral pattern on an outer surface of the shaft and extending from a leading tip portion of the shaft to a trailing portion of the shaft;
      a non-threaded buttress connecting a proximal portion of the shaft to a threaded head with a head length (L4), the head having a major diameter (M_{H}) and a minor diameter (m_{H}) with one or more screw threads of constant pitch being positioned in a spiral pattern on an outer surface of the head, such that M_{H}>M_{T} and m_{H}>m, the threaded head being adapted to accommodate a rotational tool which provides the rotational force necessary to allow rotational movement of the orthopaedic screw;
      wherein a diameter (D_{B}) of the buttress gradually increases from the proximal portion of the shaft towards the head.

In an embodiment, the one or more screw threads comprise a varying pitch that decreases from the tip portion towards the trailing portion of the shaft.

In an embodiment, the one or more screw threads comprise a constant pitch.

In an embodiment, the diameter (D_{B}) of the buttress section per unit length (x) of the buttress varies in accordance with the following equation:

D_{B}≥1.5x

In an embodiment, the compression screw comprises a canula with a first opening through the threaded head and extending through the threaded head, the buttress, the shaft and a second opening through the tip.

In an embodiment, the diameter (D_{B}) of the buttress section varies per unit length (x) of the buttress is in accordance with the following equation:

D_{B}=Ax²−Bx

wherein 0.5≤A≤1.0; and 0.05≤B≤0.1

Preferably, 0.6≤A≤0.9; and 0.06≤B≤0.09.

In an embodiment, length (L5) of the buttress section is less than 0.5 times L2.

In an embodiment, the buttress comprises at least a first buttress section connected to the shaft and at least a second buttress connected to the head such that a radius of curvature of an outer surface of the first buttress section is in the range of 0.4 mm and 2.2 mm and wherein the second buttress section comprises an inclined flat surface which is inclined towards the threaded head at an acute angle relative to a longitudinal axis of the compression screw.

In an embodiment, the outer surface of the second buttress section is inclined towards the threaded head at an angle of less than ninety degrees relative to a longitudinal axis of the elongate shaft.

In an embodiment, the outer surface of the second buttress section is inclined towards the threaded head at an angle of less than sixty degrees relative to a longitudinal axis of the elongate shaft.

In an embodiment, the outer surface of the second buttress section is inclined towards the threaded head at an angle of forty-five degrees relative to a longitudinal axis of the elongate shaft.

In an alternative embodiment, the buttress comprises at least a first buttress section connected to the shaft and at least a second buttress connected to the head such that a radius of curvature of an outer surface of the first buttress section is in the range of 0.4 mm and 2.2 mm and wherein a radius of curvature of an outer surface of the second buttress section is in the range of 0.4 mm and 0.7 mm.

In an embodiment, the minor diameter (mₜ) of the threaded section gradually decreases from the trailing portion of the threaded shaft portion to the leading portion of the threaded shaft portion.

In an embodiment, the shaft further comprises a non-threaded section having a length (L3) that extends between the proximal portion of the shaft and the trailing portion of the threaded section of the shaft.

In an embodiment, the length (L3) of the of the non-threaded section is greater than or equal to the length (L2) of the threaded section of the shaft.

In an embodiment, a diameter (DN) of the non-threaded section is less than the instant diameter (DT) across any portion of the threaded section of the shaft.

In an embodiment, the diameter (DN) of the non-threaded section is less than the diameter (DH) of the threaded head.

In an embodiment, the length (L3) of the non-threaded section is greater than length (L4) of the threaded head.

In an embodiment, the length (L3) of the non-threaded section is greater than length (L2) of the threaded section of the shaft.

In an embodiment, the threaded section of the elongate shaft comprises a double start screw thread.

In an embodiment, the threaded head comprises a double start screw thread.

In an embodiment, a shank comprises a substantially uniform crest width that does not increase with the varying pitch.

In an embodiment, ratio between the major diameter (M_{H}) of the threaded head and the major diameter (M_{T}) lies in the range 1.5:1 to 1.1:1 and more preferably in the range 1.4:1 to 1.15.

In an embodiment, the threaded head comprises a pitch that is less than an average pitch for the threaded section of the shaft.

In an alternative embodiment, the threaded head comprises a pitch that is substantially similar to the average pitch for the threaded section of the shaft.

In an embodiment, the non-threaded buttress comprises at least a first buttress section connected to the shaft and at least a second buttress connected to the head wherein at least the first buttress section comprises a featureless and even surface.

Preferably, the first buttress section comprises a length that is at least one-third the length (L5) of the non-threaded buttress. More preferably, the first buttress section comprises a length that is at least half the length (L5) of the non-threaded buttress.

### BRIEF DESCRIPTION OF THE DRAWINGS

Preferred features, embodiments and variations of the disclosure may be discerned from the following Detailed Description which provides sufficient information for those skilled in the art to perform the disclosure. The Detailed Description is not to be regarded as limiting the scope of the preceding Summary of the Disclosure in any way. The Detailed Description will make reference to a number of drawings as follows:

FIGS. 1(A) to 1(C) illustrate prior art headless compression screws.

FIG. 2 is a frontal view of the compression screw 100.

FIG. 3 is a sectional view of the compression screw 100.

FIG. 4 is another frontal view of the compression screw 100.

FIG. 4A is a frontal view of an alternative embodiment of the compression screw 100′.

FIG. 5 illustrates a method of manufacturing the compression screw 100.

FIG. 6 is an enlarged view of the buttress section 150.

FIG. 7 is a frontal view of yet another embodiment of a non-compression screw 100″.

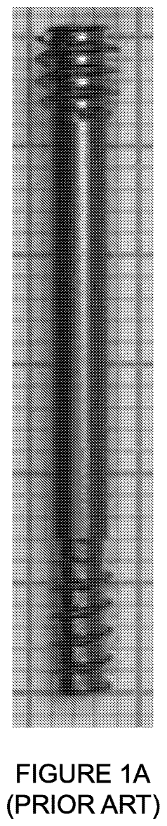

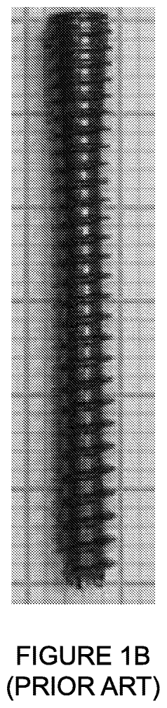

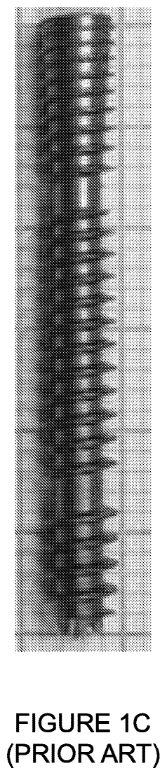

### DETAILED DESCRIPTION OF THE DRAWINGS

Throughout this disclosure mention is made of several terms which are defined herein as follows: (a) the term “lead” refers to the measurement of the distance along the screw's longitudinal axis that the screw advances relative to a fixed point upon exercising one complete revolution of the screw about its longitudinal axis; (b) the term “pitch” refers to the distance between adjacent thread crests on a screw; (c) the term “single thread” is used to denote a screw or a screw portion having a single continuous helical thread, with a single thread start, on the screw or screw portion; (d) the term “double thread” is used to denote a screw or a screw portion having two continuous helical threads, with two separate thread starts, on the screw or screw portion.

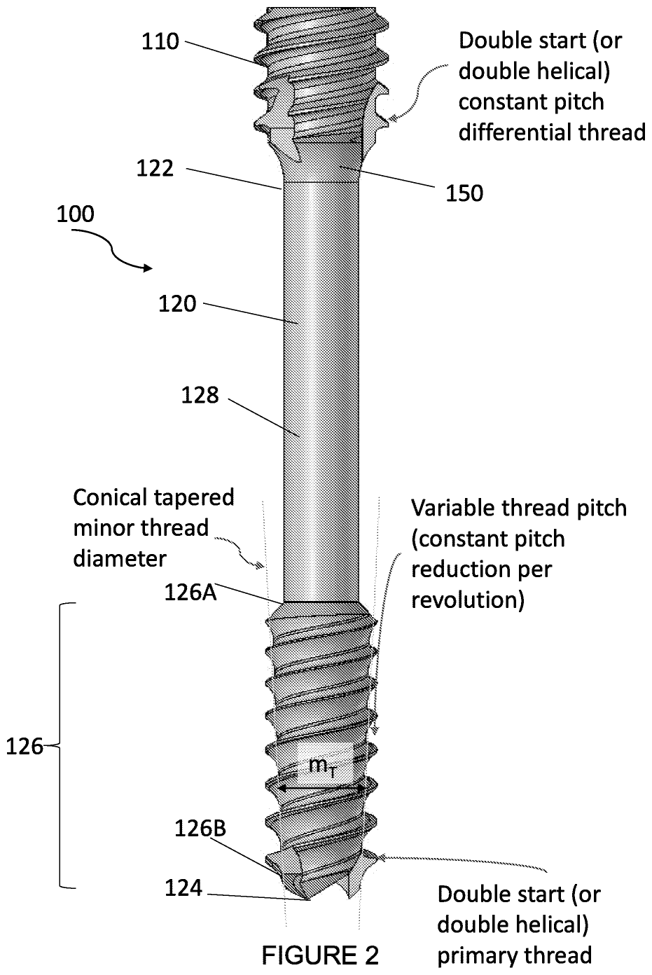

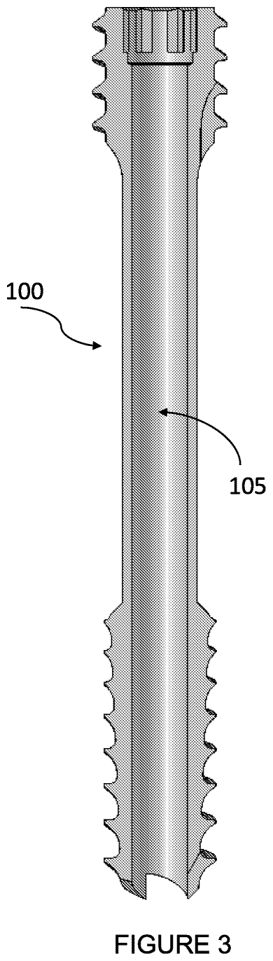

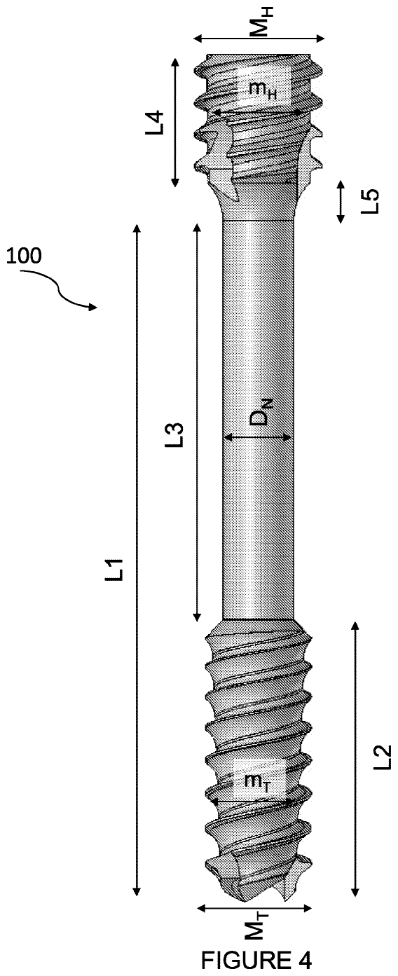

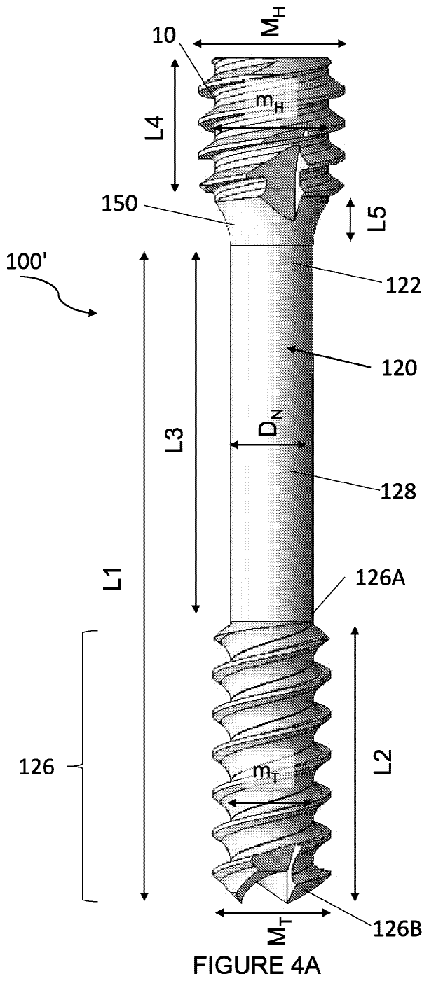

FIGS. 2 to 4 illustrate a compression screw 100 in accordance with a preferred embodiment. FIGS. 2 and 4 illustrate a frontal elevation of the compression screw 100. The compression screw 100 comprises a threaded head 110 having a major diameter (M_{H}) and a minor diameter (m) with a double start helical thread wherein the threads have a constant thread pitch. The head 110 comprises an overall length (L4) and a substantial length of the head 110 is threaded and comprises the double start helical thread. The head 110 comprises an end portion that is configured to engage a device driver to accommodate a rotational tool which provides the rotational force necessary to allow rotational movement of the orthopaedic screw. By way of example, the head 110 may be provided with a star shaped or hexagonal shaped recess.

The compression screw 100 also comprises an elongate shaft 120 having a length (L1) that extends between a proximal portion 122 (located adjacent the head 110) and a distal tip portion 124. The compression screw 100 is provided with a cannula 105 that extends along the entire length of the compression screw 100 extending coaxially through the head and the shaft with openings being provided in the head 110 and the tip 122 to allow a K-wire to be passed therethrough.

The elongate shaft 120 comprises a threaded section 126 having a length (L2) with a major diameter (M_{T}) and a minor diameter (mₜ). The threaded section 126 extends between a leading portion 126A (located at or adjacent the tip 122) and a trailing portion 126B of the threaded section 126. The threaded section 126 may comprise a double start thread with a varying pitch that decreases from the leading portion 126B towards the trailing portion 126A (as seen in FIGS. 2,3 and 4). Alternatively, in another alternative embodiment of the screw 100′ shown in FIG. 4A, the threaded section 126 may comprise a double start thread with a constant pitch (as seen in FIG. 4A). The elongate shaft 120 also comprises a non-threaded section 128 having a length (L3) which extends between the trailing section 126A of the threaded section 126 and the proximal portion 122 of the elongate shaft 120. The length (L3) of the non-threaded section 128 is preferably greater than the length (L1) of the threaded section 126.

Importantly, the dimensions of threaded head 110 are slightly larger than the elongate shaft 120. Specifically, the major diameter and minor diameter of the head are respectively greater than the threaded section. Specifically, M_{H}>M_{T} and m_{H}>m_{T}. The slightly larger dimensions of the threaded head 110 implies that the compression screw 100 described in the present embodiment does not function as a headless screw (as shown in the prior art). The provision of a slightly enlarged threaded head 110 with screw threads having a constant pitch allows the compression screw 100 to provide some advantages of a headless screw whilst also providing advantages of a headed screw. The inventors have found that having a threaded head in which the major diameter (M_{H}) of the threaded head is 1.1 to 1.5 times greater than the major diameter (M_{T}) of the threaded portion of the shaft 120. In other possible embodiments, the applicants have also found that narrowing the acceptable range for the major diameter (M_{H}) of the threaded head to be 1.2 to 1.35 times the major diameter (M_{T}) of the threaded portion of the shaft 120 also results in achieving some of the expected advantages of the presently described embodiment. As a result, the compression screw 100 can be deemed as a “hybrid” screw which bridges the gap between headless screws and headed screws. The combination of the slightly enlarged threaded head 110 in combination with a non-threaded buttress 150 discussed in further detail in the foregoing sections provides a novel buttressing effect during use.

In some embodiments, the threaded head 110 comprises threads that have a constant pitch whereby the pitch which is less than the constant pitch of the threaded shaft shown in FIG. 4A. As a result, the difference in between the pitch of the threaded head 110 and the threaded shaft in combination with the buttress 150 (that has been described in the foregoing sections) provides a compressive effect. In embodiments of the screw 100′ where the threaded shaft comprises a constant pitch, as shown in FIG. 4A, the pitch for the threads provided in the threaded section 126 of the shaft 120 must be less than the constant pitch value of the threaded section 126.

In some other embodiments where the threaded shaft 120 comprises a varying pitch, the pitch value for the threaded head 110 must not exceed the pitch value along any portion of the threaded shaft 120. Furthermore, the pitch value of the threads provided on the threaded head 110 should be less than the average pitch of the threads (with varying pitch) provided along the threaded section 126 of the shaft 120 to ensure that a compressive effect is achieved when the screw 100 is advanced through bone fragments that are to be drawn together.

Importantly, the compression screw 100 also comprises a buttress 150 connecting a proximal portion of the shaft 122 to the threaded head 110. An instantaneous diameter (D_{B}) of the buttress section 150 gradually increases from the proximal portion of the elongate shaft 122 towards the head 110 such that the rate of increase of the instantaneous diameter (D_{B}) of the buttress section 150 per unit length of the buttress is at least 1.5. The length (L5) of the buttress section 150 is less than half the length of the threaded head 110.

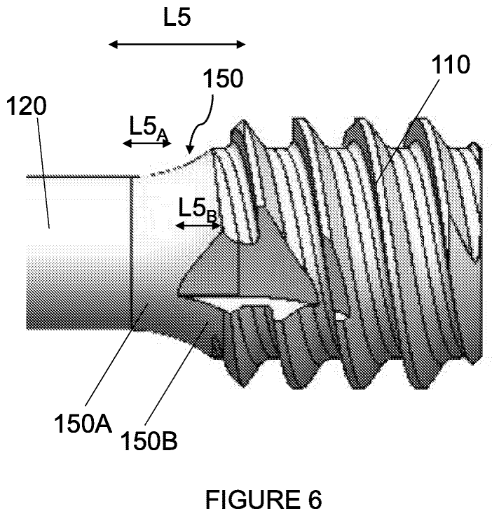

As shown most clearly in the enlarged view of FIG. 6, the buttress section 150 comprises at least a first buttress section 150A (having a length (L5_{A})) connected to the shaft 120 and at least a second buttress 150B (having a length (L5_{B})) connected to the head 110.

In a first possible embodiment, the first buttress section 150A is provided with a concavely curved outer surface. A radius of curvature of the outer surface of the first buttress section 150A may be in the range of 0.4 mm and 2.2 mm. In this first possible embodiment, the second buttress section 150B comprises an inclined flat surface which is inclined towards the threaded head 110 at an acute angle relative to a longitudinal axis of the compression screw 100. Preferably, the outer surface of the second buttress section 150B is inclined at an angle of less than 60 degrees and more preferably 45 degrees relative to the longitudinal axis of the elongate shaft 120.

In a second possible embodiment, the second buttress section 150B may also provided with a curved outer surface (instead of an inclined linear surface in the first possible embodiment) such that a radius of curvature of an outer surface of the second buttress section 150B is in the range of 0.4 mm and 0.7 mm.

In one particular embodiment, the diameter (D_{B}) of the buttress section per unit length (x) of the buttress in a direction towards the head 110 increases as shown below.

| X | D_{B} |
|---|---|
| 0 | 0 |
| 0.026179 | 0.000228 |
| 0.052349 | 0.000914 |
| 0.078504 | 0.002056 |
| 0.104635 | 0.003654 |
| 0.130734 | 0.005708 |
| 0.156793 | 0.008217 |
| 0.182804 | 0.011181 |
| 0.20876 | 0.014598 |
| 0.234652 | 0.018467 |
| 0.260472 | 0.022788 |
| 0.286213 | 0.027559 |
| 0.311868 | 0.032779 |
| 0.337427 | 0.038445 |
| 0.362883 | 0.044556 |
| 0.388229 | 0.051111 |
| 0.413456 | 0.058107 |
| 0.438558 | 0.065543 |
| 0.463525 | 0.073415 |
| 0.488352 | 0.081722 |
| 0.51303 | 0.090461 |
| 0.537552 | 0.099629 |
| 0.56191 | 0.109224 |
| 0.586097 | 0.119243 |
| 0.610105 | 0.129682 |
| 0.633927 | 0.140538 |
| 0.657557 | 0.151809 |
| 0.680986 | 0.16349 |
| 0.704207 | 0.175579 |
| 0.727214 | 0.18807 |
| 0.75 | 0.200962 |
| 0.772557 | 0.214249 |
| 0.794879 | 0.227928 |
| 0.816959 | 0.241994 |
| 0.838789 | 0.256444 |
| 0.860365 | 0.271272 |
| 0.881678 | 0.286475 |
| 0.902723 | 0.302047 |
| 0.923492 | 0.317984 |
| 0.943981 | 0.334281 |
| 0.964181 | 0.350933 |
| 0.984089 | 0.367936 |
| 1.003696 | 0.385283 |
| 1.022998 | 0.402969 |
| 1.041988 | 0.42099 |
| 1.06066 | 0.43934 |

The inventors have found that in some embodiments, screws having a cannula diameter of up to 3.0 mm, the changing diameter can be defined in accordance with the following function:

$$2 [ - 0.133 {x}^{4} + 0.3761 {x}^{3} + 0.096 {x}^{2} + 0.0422 x ] .$$

The above equation may be simplified as follows:

$$2 [ 0.4088 {x}^{2} - 0.0322 x ]$$

In other embodiments, screws having a cannula diameter of up to 3.0 mm, the changing diameter can be defined in accordance with the following function

$$2 [ - 0.0474 {x}^{4} + 0.1874 {x}^{3} + 0.0925 {x}^{2} + 0.0371 x ]$$

The above equation may be simplified as follows:

$$2 [ 0.3172 {x}^{2} - 0.043 x ]$$

Through extensive experimentation involving the use of finite element analysis (FEA) and novel modelling that involved examining the stresses acting in the buttress section 150, the inventors have somewhat surprisingly found that providing a buttress in the any of the aforementioned configurations above provides a significant improvement and reduced stress concentration in the buttress section 150 of the screw 100. In addition to improved mechanical characteristics, the buttress section 150 is provided with a wider and progressively increasing diameter to allow external buttressing as the variable thread in the threaded section 126 compresses first and second bone fragments during use. The buttress section 150 provides an outer surface against which the first bone fragment can drive and compress against the second bone fragment thereby providing external compression in addition to the compression provided by the threaded section 126 of the elongate shaft 120.

The first buttress section 150A (having a length (L5_{A})) connected to the shaft 120 preferably comprise a substantially featureless and smooth surface. Cutting flutes may be provided in the second buttress 150B (having a length (L5_{B})) connected to the head 110. It is also preferable to have the length L5_{A} of the first buttress section 150A to be at least ⅓^{rd} and preferably at least ½ the length L5_{B} of the second buttress section 150B.

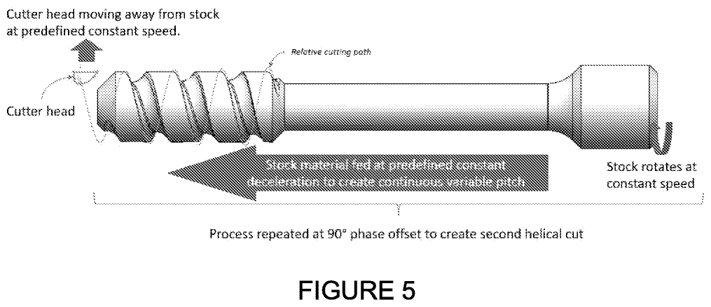

We now refer to FIGS. 2 and 4 in particular, which shows the threaded section 126 of the elongate shaft 120. The double start threads on the threaded section 126 comprise a varying pitch that that decreases from the tip portion 126B towards the trailing portion of the shaft 126A which allows bone fragments engaging these threads to be compressed towards each other. The major diameter M_{T} is substantially constant and does not vary or taper. The minor diameter my on the other hand tapers inwardly from the trailing portion 126A towards the tip portion 126B. FIG. 4A illustrates an alternative embodiment of the screw 100′ wherein the threaded section 126 comprises a constant pitch. FIG. 5 illustrates the manner in which the variable pitch for the threaded section 126 is achieved. The variable pitch thread for the threaded section 126 is made by not only varying the feed rate of the stock material (fed at constant deceleration but rotated at a constant speed), but also compensating by moving the cutter head away from the stock material (as shown in FIG. 5) which results in the variation of the minor diameter m_{T}. If this compensation was not made, the thread profile would be unusual because of the variable pitch, resulting in the outer diameter of the thread having a ‘crest width’ that increases progressively. In such a scenario of increasing crest width, the outer thread along the distal sections of the threaded section 126 would get very blunt because the relatively sharp part of the thread gets wider (and blunt). By adopting this methodology, the crest width of the threads is maintained at a constant crest width.

In the embodiment shown in FIG. 4, due to the decreasing pitch along the length of the screw 100, each successive thread received in the path cut by the thread exerts pressure against the side of the path cut by the thread thereby tending to compress the bone along the length of the screw 100. As a result, by the time the screw 100 is fully installed, the trailing thread compresses a substantial amount of bone when it is received in the path cut by the leading thread. This tends to draw bone fragments tightly together across fractures thereby promoting healing of the fracture. In contrast, the screw 100 having a threaded section 126 comprising a constant pitch (as seen in FIG. 4A) only provides compression from the non-threaded buttress 150 and threaded head 110.

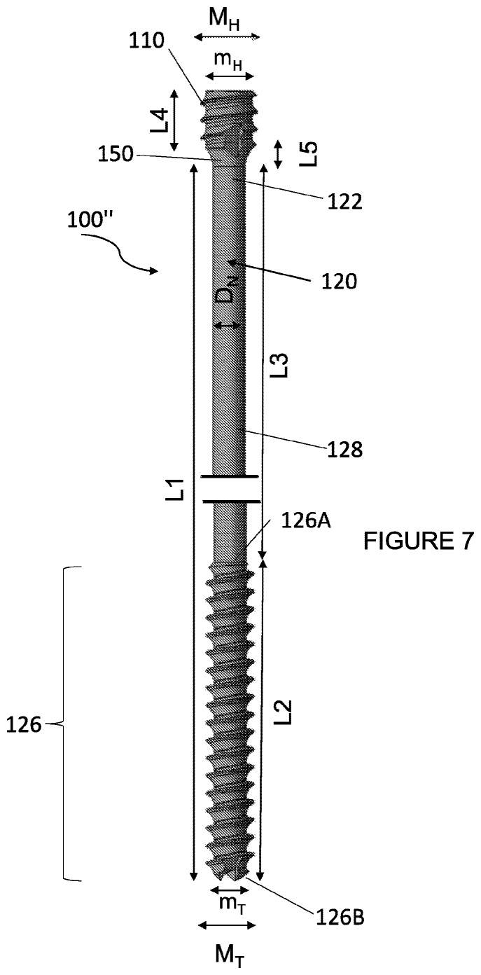

FIG. 7 illustrates another embodiment of a non-compression screw 100″. Like reference numerals denote like features that have previously been discussed.

The non-compression screw 100″ comprises a threaded head 110 having a major diameter (M_{H}) and a minor diameter (m_{H}) with a double start helical thread wherein the threads have a constant thread pitch. The head 110 comprises an overall length (L4) and a substantial length of the head 110 is threaded and comprises the double start helical thread. The head 110 comprises an end portion that is configured to engage a device driver to accommodate a rotational tool which provides the rotational force necessary to allow rotational movement of the orthopaedic screw. By way of example, the head 110 may be provided with a star shaped or hexagonal shaped recess.

The non-compression screw 100 also comprises an elongate shaft 120 having a length (L1) that extends between a proximal portion 122 (located adjacent the head 110) and a distal tip portion 124. The non-compression screw 100 is also provided with a cannula 105 that extends along the entire length of the compression screw 100 extending coaxially through the head and the shaft with openings being provided in the head 110 and the tip 122 to allow a K-wire to be passed therethrough.

The elongate shaft 120 comprises a threaded section 126 having a length (L2) with a major diameter (M_{T}) and a minor diameter (mₜ). The threaded section 126 extends between a leading portion 126A (located at or adjacent the tip 122) and a trailing portion 126B of the threaded section 126. The threaded section 126 may comprise a double start thread with a constant pitch extending from the leading portion 126B towards the trailing portion 126A The elongate shaft 120 also comprises a non-threaded section 128 having a length (L3) which extends between the trailing section 126A of the threaded section 126 and the proximal portion 122 of the elongate shaft 120. The length (L3) of the non-threaded section 128 is preferably greater than the length (L1) of the threaded section 126.

Once again, the dimensions of threaded head 110 are slightly larger than the elongate shaft 120. Specifically, the major diameter and minor diameter of the head are respectively greater than the threaded section. Specifically, M_{H}>M_{T} and m_{H}>m_{T}. The slightly larger dimensions of the threaded head 110 implies that the non-compression screw 100″ described in the present embodiment does not function as a headless screw (as shown in the prior art). The provision of a slightly enlarged threaded head 110 with screw threads having a constant pitch allows the non-compression screw 100″ to provide some advantages of a headless screw whilst also providing advantages of a headed screw. The inventors have found that having a threaded head in which the major diameter (M_{H}) of the threaded head is 1.1 to 1.5 times greater than the major diameter (M_{T}) of the threaded portion of the shaft 120 can provide some key advantages. In other possible embodiments, the applicants have also found that narrowing the acceptable range for the major diameter (M_{H}) of the threaded head to be 1.2 to 1.35 times the major diameter (M_{T}) of the threaded portion of the shaft 120 also results in achieving some of the expected advantages of the presently described embodiment. As a result, the non-compression screw 100 can be deemed as a “hybrid” screw which bridges the gap between headless screws and headed screws.

In some embodiments, the threaded head 110 in the non-compression screw 100″ comprises threads that have a constant pitch whereby the pitch for these screw threads in the head 110 is substantially the same as the pitch for the screw threads provided on the threaded section 126 of the shaft 120. Due to the similarity in the thread pitch between the head 110 and the threaded shaft section 126 there is no compressive effect. However, the provision of the non-threaded buttress 150 as previously described still provides a beneficial buttressing effect.

In compliance with the statute, the disclosure has been described in language more or less specific to structural or methodical features. The term “comprises” and its variations, such as “comprising” and “comprised of” is used throughout in an inclusive sense and not to the exclusion of any additional features.

It is to be understood that the disclosure is not limited to specific features shown or described since the means herein described comprises preferred forms of putting the disclosure into effect.

The disclosure is, therefore, claimed in any of its forms or modifications within the proper scope of the appended claims appropriately interpreted by those skilled in the art.

1. A compression screw comprising:
an elongate shaft having a shaft length extending between a proximal portion and a distal tip portion, the shaft comprising:
a threaded section of the shaft having a length with a major diameter and a minor diameter, the threaded section having one or more screw threads positioned in a spiral pattern on an outer surface of the shaft and extending from a leading tip portion of the shaft to a trailing portion of the shaft;
a non-threaded buttress connecting a proximal portion of the shaft to a threaded head with a head length, the head having a major diameter and a minor diameter with one or more screw threads of constant pitch being positioned in a spiral pattern on an outer surface of the head, such that M_{H}>M_{T} and m_{H}>m_{T}, the threaded head being adapted to accommodate a rotational tool which provides the rotational force necessary to allow rotational movement of the orthopaedic screw;
wherein a diameter of the buttress gradually increases from the proximal portion of the shaft towards the head.

2. The compression screw in accordance with claim 1 wherein the one or more screw threads comprise a varying pitch that decreases from the tip portion towards the trailing portion of the shaft.

3. The compression screw in accordance with claim 1 wherein the one or more screw threads comprise a constant pitch.

4. The compression screw in accordance with claim 1 wherein the rate of increase of the diameter of the buttress section per unit length of the buttress is in accordance with the following equation:
$${D}_{B} \geq 1.5 x .$$

5. The compression screw in accordance with claim 1 wherein such the diameter of the buttress section varies per unit length of the buttress is in accordance with the following equation:
$${D}_{B} = A {x}^{2} - B x$$
wherein 0.5≤A≤1.0; and 0.05≤B≤0.1

6. The compression screw in accordance with claim 5 wherein 0.6≤A≤0.9; and 0.06≤B≤0.09.

7. The compression screw in accordance with claim 1 wherein length of the buttress section is less than 0.5 times L2.

8. The compression screw in accordance with claim 1 comprising a canula with a first opening through the threaded head and extending through the threaded head, buttress, and shaft and a second opening through the tip.

9. The compression screw in accordance with claim 1 wherein the buttress comprises at least a first buttress section connected to the shaft and at least a second buttress connected to the head such that a radius of curvature of an outer surface of the first buttress section is in the range of 0.4 mm and 2.2 mm and wherein the second buttress section comprises an inclined flat surface which is inclined towards the threaded head at an acute angle relative to a longitudinal axis of the compression screw.

10. The compression screw in accordance with claim 9 wherein the outer surface of the second buttress section is inclined towards the threaded head at an angle of less than ninety degrees relative to a longitudinal axis of the elongate shaft.

11. The compression screw in accordance with claim 9 wherein the outer surface of the second buttress section is inclined towards the threaded head at an angle of less than sixty degrees relative to a longitudinal axis of the elongate shaft.

12. The compression screw in accordance with claim 9 wherein the outer surface of the second buttress section is inclined towards the threaded head at an angle of forty-five degrees relative to a longitudinal axis of the elongate shaft.

13. The compression screw in accordance with claim 1 wherein the buttress comprises at least a first buttress section connected to the shaft and at least a second buttress connected to the head such that a radius of curvature of an outer surface of the first buttress section is in the range of 0.4 mm and 2.2 mm and wherein a radius of curvature of an outer surface of the second buttress section is in the range of 0.4 mm and 0.7 mm.

14. The compression screw in accordance with claim 1 wherein the minor diameter of the threaded section gradually decreases from the trailing portion of the threaded shaft portion to the leading portion of the threaded shaft portion.

15. The compression screw in accordance with claim 1 wherein the shaft further comprises a non-threaded section having a length that extends between the proximal end portion of the shaft and the trailing portion of the threaded section of the shaft.

16. The compression screw in accordance with claim 10 wherein the length of the of the non-threaded section is greater than or equal to the length of the threaded section of the shaft.

17. The compression screw in accordance with claim 12 wherein a diameter of the non-threaded section is less than the instant diameter across any portion of the threaded section of the shaft.

18. The compression screw in accordance with claim 14 wherein the diameter of the non-threaded section is less than the diameter of the threaded head.

19. The compression screw in accordance with claim 12 wherein the length of the non-threaded section is greater than length of the threaded head.

20-28. (canceled)

29. A non-compression screw comprising:
an elongate shaft having a shaft length extending between a proximal portion and a distal tip portion, the shaft comprising:
a threaded section of the shaft having a length with a major diameter and a minor diameter, the threaded section having one or more screw threads positioned in a spiral pattern on an outer surface of the shaft and extending from a leading tip portion of the shaft to a trailing portion of the shaft;
a non-threaded buttress connecting a proximal portion of the shaft to a threaded head with a head length, the head having a major diameter and a minor diameter with one or more screw threads of constant pitch being positioned in a spiral pattern on an outer surface of the head, such that M_{H}>M_{T} and m_{H}>m_{T}, the threaded head being adapted to accommodate a rotational tool which provides the rotational force necessary to allow rotational movement of the orthopaedic screw;
wherein a diameter of the buttress gradually increases from the proximal portion of the shaft towards the head.

30-55. (canceled)
